(19) United States
(12) Patent Application Publication
(10) Pub. No.: US 2011/0269223 A1
(43) Pub. Date: Nov. 3, 2011

(54) CORROSION INHIBITION

(75) Inventors: Torbjörn Van Der Meulen, Ornskoldsvik (SE); Gunnar Fransson, Burea (SE); Lars Sundlöf, Skelleftea (SE); Jan Lindstedt, Ornskoldsvik (SE)

(73) Assignee: SEKAB E-TECHNOLOGY AB, Ornskoldsvik (SE)

(21) Appl. No.: 13/062,286

(22) PCT Filed: Sep. 7, 2009
(86) PCT No.: PCT/SE2009/050998

§ 371 Date: May 13, 2011

(30) Foreign Application Priority Data
Sep. 5, 2008 (SE) 0801913-5

(51) Int. Cl. C12M 1/00; C23F 11/00; F15D 1/00; B01J 19/00

(52) U.S. Cl. 435/289.1; 422/40; 422/7; 137/1

(57) ABSTRACT

The present invention relates to a methods, products and uses for inhibiting corrosion. A provided method comprises the steps of: a) subjecting cellulosic biomass to hydrolysis to obtain a hydrolysate liquid; b) optionally, subjecting the hydrolysate liquid from step a) to one or more of the steps of further hydrolysis, fermentation and/or distillation; and c) using the hydrolysate liquid from step a) or b) to inhibit corrosion.

### TECHNICAL FIELD OF THE INVENTION

The present invention relates to corrosion inhibition, in particular in industrial processes.

### BACKGROUND ART

In many industrial processes, metallic surfaces are subjected to corrosive fluids that negatively affect the lifetime of the materials and the efficiency of the process. One such industrial process is the extraction of saccharides from cellulosic biomass, e.g. when producing ethanol. When processing cellulosic biomass, several treatment processes are often used. Such processes include acid hydrolysis to extract fermentable saccharides from the cellulose and hemicellulose contained in the cellulosic biomass. The acid solutions are strongly corrosive and thus represent a danger for the surfaces and other materials that contact the solutions. Moreover, the acid hydrolysis of cellulosic biomass is commonly performed at high temperature, which also contributes to a high rate of corrosion. Corrosion of materials used when processing cellulosic biomass both lead to high material costs and the formation of metal ions, which may be seriously inhibiting in the subsequent process steps, such as the fermentation of extracted sugars into ethanol.

A common strategy for preventing corrosion during acid hydrolysis is to use hydrolysis vessels and peripheral equipment of high-quality materials having a high corrosion resistance. For example, hydrolysis vessels have been manufactured entirely from zirconium, which is an extremely expensive material. Other examples of construction materials for acid hydrolysis include titanium and titanium alloys, beryllium, or different alloys with nickel as a major constituent. However, all of these materials are very expensive and thus hampers the industrial usage.

Another strategy for preventing or inhibiting corrosion in industrial processes is to use anti-corrosive agents, i.e. compounds added to the process with the aim to decrease the rate or extent of corrosion. The compounds may be added onto the material exposed to the corrosive fluid or to the fluid itself. Examples of anti-corrosive agents used are e.g. anodic inhibitors and cathodic inhibitors. However, it may be difficult to select the right type of anti-corrosive agent, since the suitability depends on several factors, such as the materials used in the processes, the operating temperature of the process and the properties of the corrosive fluid the agent is added into. The added anti-corrosive agent may also interfere with the industrial process and lead to other negative side effects, such as inhibition of subsequent processes.

### SUMMARY OF THE INVENTION

It is an aim of some aspects of the present disclosure to provide means or a method for inhibiting corrosion.

It is a further aim of some aspects the present disclosure to reduce the material costs or material consumption when hydrolysing cellulosic biomass.

The present invention is defined by the appending claims.

As a first aspect of the invention, there is provided a method for inhibiting corrosion comprising the steps of:
a) subjecting cellulosic biomass to hydrolysis to obtain a hydrolysate liquid;
b) optionally, subjecting the hydrolysate liquid from step a) to one or more of the steps of further hydrolysis, fermentation and/or distillation;
c) using the hydrolysate liquid from step a) or b) to inhibit corrosion.

In the context of the present disclosure, corrosion refers to the breaking down or the destruction of a material, especially a metal, through chemical reactions. Consequently, the term “inhibiting corrosion” refers to decreasing the rate of the chemical reaction leading to corrosion, thereby decreasing or eliminating the extent of the corrosion. The inhibiting of corrosion may thus involve any mechanisms that affect the various electrical parameters contributing to the overall corrosion reaction so that the rate and/or extent of the corrosion is decreased. The inhibiting of corrosion may comprise the altering of the electrical conductivity of a corrosive fluid, such as the neutralization of active ions, the reduction of ion mobility and/or the changing of ion transport numbers.

Cellulosic biomass refers to biological material comprising cellulose. Without being limited thereto, the cellulosic biomass of step a) may be selected from wood residues, municipal waste, agricultural residues, such as bagasse, and energy crops. These sources of cellulosic biomass are abundant raw materials with the potential to give a high net energy gain. The wood residues may be forestry residues, such as wood chips, sawmill or paper mill discards. The municipal waste may be paper waste, e.g. recycled paper or paperboard. Agricultural residues may be corn stover, corn fiber, wheat straw, sugarcane bagasse, beet pulp, rice straw or soybean stover and energy crops may be fast growing trees or woody grasses. Other sources of cellulosic biomass are well known to the skilled person.

In general, hydrolysis refers to a chemical reaction in which water reacts with a compound to produce other compounds. In the context of the present disclosure, a hydrolysate liquid refers to the product obtained after one or several process steps, wherein at least one process step involves hydrolysis, preferably hydrolysis of cellulose, such as lignocellulose. Thus, the hydrolysate liquid may be a hydrolysate, which may comprise saccharides, or the hydrolysate liquid may be a fermentation broth obtained from fermentation of a hydrolysate comprising saccharides, or the hydrolysate liquid may be a stillage obtained after distillation of such fermentation broth. It may be beneficial to use the stillage instead of the fermentation broth or the hydrolysate since the stillage is generally considered to be of lower value.

Further, hydrolysis of cellulosic biomass refers to the depolymerisation of polymers comprised in the cellulosic biomass, such as the depolymerisation of cellulose and/or hemicellulose.

Subjecting the hydrolysate liquid to optional further hydrolysis in step b) refers to subjecting the hydrolysate liquid obtained from step a) to at least one more hydrolysis process step. Such a process step may be a hydrolysis in different environmental conditions compared to the hydrolysis of step a), such as a hydrolysis at a different temperature, at a different pH and/or at a different pressure compare to the hydrolysis of step a). Moreover, the further hydrolysis may be a similar hydrolysis process as the hydrolysis of step a). Also, the further hydrolysis may be several subsequent hydrolysis process steps. The further hydrolysis may be at least one acid hydrolysis process, and/or at least one enzymatic hydrolysis comprising the use of saccharification enzymes, i.e. enzymes able to convert components of the hydrolysate into free sugars, or a combination of acid hydrolysis and enzymatic hydrolysis.

Subjecting the hydrolysate liquid to optional fermentation in step b) refers to subjecting a hydrolysate liquid obtained from step a) or the further hydrolysis to conditions in which chemicals extracted from the cellulosic biomass and comprised in the hydrolysate liquid are fermented into a target product. As an example, fermentation may be the use of microorganisms, such as yeast, to convert saccharides comprised in the hydrolysate into ethanol.

Subjecting the hydrolysate liquid to distillation refers to subjecting the hydrolysate liquid obtained from the optional fermentation to a separation in which at least one type of chemical comprised in the hydrolysate liquid is separated from the hydrolysate liquid. As an example, distillation may be the separation of ethanol from a hydrolysate liquid that has been fermented by microorganisms.

The first aspect of the invention is based on the insight that a hydrolysate liquid obtained after at least one hydrolysis step of cellulosic biomass can surprisingly be used to inhibit corrosion. Consequently, hydrolysate liquid obtained from cellulosic biomass may facilitate the use of non-exclusive material in industrial processes, thus decreasing the material cost or consumption in a process that otherwise require exclusive materials of high costs. Further, the hydrolysate liquid obtained from cellulosic biomass may reduce or eliminate the need of adding external anti-corrosive agents, which may interfere with the desired process.

In an embodiment of the first aspect, step c) comprises adding the hydrolysate liquid in a process to inhibit corrosion of one or more apparatuses used in the process.

If the hydrolysate liquid obtained from the cellulosic biomass is used to inhibit corrosion in an apparatus used in the same process, the lifetime of the apparatus increase and the apparatus may be constructed of a less exclusive material. The apparatus may be a metallic apparatus and may comprise containers, vessels or pipes or be used as containers, vessels or pipes. According to one embodiment, the hydrolysate liquid of step c) is added as at least part of a process fluid in the process, and the one or more apparatuses are in contact with the process fluid.

Additional concentration etc. of the hydrolysate liquid before using it as a corrosion inhibitor is not required, but may be performed in some embodiments. Further, the hydrolysate liquid may be used as such or be diluted with other fluids before being used to inhibit corrosion.

In one embodiment, the pH of the process fluid is 0-6, such as 0.5-4.5, such as 0.5-3, such as 0.5-2. Process fluids of the disclosed pH-intervals may be commonly used in the processing of cellulosic biomass.

According to an embodiment, the one or more apparatuses comprise iron.

The one or more apparatuses may be containers used in different process steps, reaction vessels, material conveyors or pipes. Apparatuses comprising iron may be extra sensitive to corrosion, and therefore, the use of a hydrolysate liquid to inhibit corrosion facilitates the use of such apparatuses.

According to an embodiment, the one or more apparatuses comprise iron in an amount of about 50% or more by weight.

In general, the greater amount iron a material comprises, the lower the production cost of the material is. A material comprising iron in an amount of about 50% or more by weight is useful in several industrial processes.

In an embodiment of the first aspect, the one or more apparatuses comprise iron in an amount of 50-75% by weight, chromium in an amount of 10.5-30% by weight, nickel in an amount of 2.5-29% by weight and molybdenum in an amount of 0-7% by weight.

In another embodiment, the one or more apparatuses comprise iron in an amount of 55-70% by weight, chromium in an amount of 15-26% by weight, nickel in an amount of 5-10% by weight and molybdenum in an amount of 1.5-5.5% by weight.

Materials comprising iron, chromium, nickel and molybdenum have been found to be useful, due to easy welding of the materials and low production costs.

In one embodiment, the one or more apparatuses are made of stainless steel.

Stainless steel is referred to as a steel alloy comprising chromium. The stainless steel may have a content of chromium above 8%, such as above 9%, such as above 10%, such as above 10.5%, such as above 11.5%. The stainless steel may be austenitic stainless steels. The austenitic stainless steel may comprise iron in an amount of at least 40% by weight, chromium in an amount of 12-30% by weight, nickel in an amount of 7-29% by weight as well as some other metals or substances, such as molybdenum in 2-3% by weight. The content of carbon in the stainless steels are low, generally below 0.05%, making the stainless steels relatively simple to machine mechanically or to weld into e.g. pipe conduits. The stainless steel may also be acid-proof stainless steel.

In one embodiment, the stainless steel is ferrite-austenitic stainless steel. Ferrite-austenitic stainless steels, also called duplex stainless steels, may comprise iron in an amount of at least 50% by weight, chromium up to 29% by weight, nickel in an amount of 5-8% by weight, molybdenum in an amount of 1-4% by weight, carbon in an amount of below 0.03% by weight and nitrogen in an amount of about 0.4% by weight. Ferrite-austenitic stainless steels may have high strength and mechanical strength combined with a good workability and weldability. Hydrolysate liquids have shown to inhibit the corrosion of ferrite-austenitic stainless steels, i.e. duplex stainless steels, as shown in Examples 1-2 in the present disclosure.

In one embodiment the stainless steel is of the grade EN 1.4462 or EN 1.4410. EN refers to the European standard grade of stainless steel. The EN 1.4462 stainless steel may have a composition in weight % of typically: phosphorous=0.02, silicon=1.0, carbon=0.02, chromium=22, nitrogen=0.17, manganese=0.5, molybdenum=3.1, nickel=5.5, sulphur<0.01, iron=67.7. The EN 1.4410 stainless steel may have a composition in weight % of typically: phosphorous=0.02, silicon=1.2, carbon=0.02, chromium=25, nitrogen=0.28, manganese=0.3, molybdenum=3.9, nickel=7, sulphur=0.002, iron=62.3.

According to an embodiment of the first aspect, the hydrolysate liquid of step c) is added in a step i), which is performed upstream of step a).

A process performed upstream of step a) refers to all processes that are performed before step a) in the processing of the cellulosic biomass. Thus, the hydrolysate liquid may be recirculated and be used to inhibit corrosion in a process performed before the hydrolysis of the cellulosic biomass, which reduces the use of e.g. fresh water and lowers the production of wastewater in the whole process.

In one embodiment, step i) comprises pretreatment of the cellulosic biomass. Pretreatment of the cellulosic biomass refers to the process of improving the formation of sugars or the ability to form them during succeeding hydrolysis. As an example, pretreatment may decrease the degree of crystallinity of the cellulosic biomass. The pretreatment may increase the porosity of the cellulosic biomass. If lignocellulosic biomass is used, pretreatment may be performed by using a fluid under conditions so that cellulose and hemicellulose is liberated from lignin. Step i) may involve one or several pretreatment methods known to the skilled man.

In one embodiment, the pretreatment is impregnation. Impregnation refers to impregnating the cellulosic biomass with an impregnation fluid. The fluid may be an acid solution, such as a mineral acid solution. The impregnation may be performed with acid solutions having different pH, such as a pH of 0.5-5.5, or such as a pH of 0.5-2. The pH of the acid solution used for impregnation may depend on the type of cellulosic biomass used. As an example, if bagasse is used, an acid solution used for impregnation may have a pH of 2-5. Also, if a wood-derived cellulosic biomass, such as a pine residue, is used, an acid solution used for impregnation may have a pH of 0.5-2.5. The impregnation may also be performed with a gas, such as a SO₂-gas, or with the combination of a gas with a liquid. The pretreatment may also be steaming or steaming followed by impregnation. Steaming refers to a process used to drive air out from the cellulosic biomass to facilitate further hydrolysis of the cellulose. Steaming is a well-known method for pretreating e.g. lignocellulosic biomass. As other examples, pretreatment may involve prehydrolysis, impregnation, steaming, steam explosion or any combination thereof. Steam explosion refers to a process that combines steam, shearing forces and hydrolysis for rupturing cellulosic fibers.

In one embodiment of the first aspect, the hydrolysate liquid, optionally after purification, e.g. by filtration, is mixed with an acid to form a pretreatment liquid which is added in step i).

Such filtration may for example remove solid lignin. It is submitted that the inventors believe that solid lignin in the hydrolysate liquid is not the source of the anti-corrosive effect. This is supported by the examples below, in which filtrates are used. The method of the first aspect may thus comprise a step of removal of solid lignin. In addition to filtration, lignin may for example be removed by sedimentation.

According to an embodiment of the first aspect, the cellulosic biomass is lignocellulosic biomass. Lignocellulosic biomass refers to biomass that comprises cellulose, hemicellulose and lignin. The lignocellulosic biomass may for example be wood residues or forestry residues, such as wood chips, sawmill or paper mill discards, or agricultural residues, such as bagasse.

In one embodiment of the first aspect, step a) and/or the further hydrolysis of step b) comprise subjecting the cellulosic biomass to an acid hydrolysis. Acid hydrolysis refers to hydrolysis with a liquid having a low pH, such as a pH of below 5, such as pH of below 4, such as pH of below 3, such as pH of below 2. Liquids of low pH, such as acidic solutions, are known to be effective for breaking linkages, e.g. the linkages between lignin and cellulose and the linkages between lignin and hemicellulose, thus allowing good accessibility of the cellulose to further processing. Further, the acid solutions are also known to depolymerise cellulose.

In one embodiment, step a) and/or the further hydrolysis of step b) comprise subjecting the cellulosic biomass to an acid hydrolysing liquid to which a mineral acid, such as H₂SO₄, and/or SO₂ gas has been added. Mineral acids, such as H₂SO₄, and SO₂ gas are well known to generate acidic liquids having a low pH. Other mineral acids that may be used are hydrochloric acid, nitric acid, phosphoric acid, boric acid and hydrofluoric acid.

In one embodiment, H₂SO₄ is of a concentration of 0.6-1.0% by weight in the acid hydrolysing liquid.

In one embodiment, the acid hydrolysis comprises subjecting the cellulosic biomass to a solution having a pH of 1.5-2.3.

A solution having a pH of 1.5-2.3 is known to be effective when hydrolysing cellulosic biomass.

In one embodiment, the acid hydrolysis is performed at a temperature of 160-240° C. In an embodiment, the acid hydrolysis is performed at a pressure of 6-34 bar. In another embodiment, the acid hydrolysis is performed during 1-60 min. Performing an acid hydrolysis at high temperatures, such as at a temperature of 160-240° C. and at high pressures, such as at a pressure of 6-34 bar, and during 1-60 min is known to increase the hydrolysis rate of cellulosic biomass in an acid hydrolysis process.

In one embodiment of the first aspect, the further hydrolysis of step b) comprises subjecting the hydrolysate to a solution having a pH of 1.5-2.3 at a temperature above 200° C. and a pressure above 19 bar.

In another embodiment, the hydrolysis of step a) comprises hydrolysing the cellulosic biomass in a solution having a pH of 1.5-2.3 at a temperature above 200° C. and a pressure above 19 bar.

In yet another embodiment, the hydrolysis of step a) comprises hydrolysing the cellulosic biomass in a solution having a pH of 1.5-2.3 at a temperature of 160-200° C. and a pressure of 8-12 bar and the further hydrolysis of step b) comprises hydrolysing the cellulosic biomass in a solution having a pH of 1.5-2.3 at a temperature above 200° C. and a pressure above 19 bar.

In another embodiment of the first aspect, the fermentation of step b) comprises subjecting the hydrolysate liquid to a microorganism, which converts sugar to ethanol. The microorganism may be bacteria fungi and/or yeast, e.g. a yeast capable of fermenting saccharides into ethanol. The ethanol-producing yeast may be a strain of Saccaromyces cerevisiae.

In another embodiment, the distillation of step b) comprises separating fermentation products from the hydrolysate liquid. The fermentation product may be ethanol and the distillation may involve separation of ethanol from the hydrolysate liquid on the basis of differences in boiling points. As an example, if the fermentation product is ethanol, distillation is a preferred method for separating ethanol from the hydrolysate liquid due to the lower boiling point of ethanol compared to the other substances comprised in the hydrolysate liquid.

The hydrolysate liquid may be subjected to work up before being used for prevention of corrosion. Examples of such a work up are filtration and/or concentration of the hydrolysate liquid. The concentration may be particularly suitable when the hydrolysate liquid is to be used for corrosion prevention in another situation/context than the process in which the hydrolysate liquid is produced. As mentioned above, the filtration may for example remove solid residues of lignin.

As a first configuration of a second aspect of the invention, there is provided a hydrolysate liquid obtainable by a process comprising the steps of:
a) providing cellulosic biomass;
b) impregnating the cellulosic biomass in a wood/liquid ratio of about from 1:1 to 1:7 to provide an impregnated cellulosic biomass and
c) hydrolysing the impregnated cellulosic biomass in a solution having a pH of 1.5-2.3 at a temperature of 160-200° C. and a pressure of 8-12 bar to provide the hydrolysate liquid.

As a second configuration of the second aspect of the invention, there is provided a hydrolysate liquid obtainable by a process comprising the steps of:
a) providing cellulosic biomass;
b) impregnating the cellulosic biomass in a wood/liquid ratio of about from 1:1 to 1:7 to provide an impregnated cellulosic biomass;
c) hydrolysing the impregnated cellulosic biomass in a solution having a pH of 1.5-2.3 at a temperature of 160-200° C. and a pressure of 8-12 bar to provide a hydrolysed cellulosic biomass and
d) further hydrolysing the hydrolysed cellulosic biomass in a solution having a pH of 1.5-2.3 at a temperature above 200° C. and a pressure above 19 bar to provide the hydrolysate liquid.

As a third configuration of the third aspect of the invention, there is provided a hydrolysate liquid obtainable by a process comprising the steps of:
a) providing cellulosic biomass;
b) impregnating the cellulosic biomass in a wood/liquid ratio of about from 1:1 to 1:7 to provide an impregnated cellulosic biomass;
c) hydrolysing the impregnated cellulosic biomass in a solution having a pH of 1.5-2.3 at a temperature of 160-200° C. and a pressure of 8-12 bar to provide a hydrolysed cellulosic biomass;
d) further hydrolysing the hydrolysed cellulosic biomass in a solution having a pH of 1.5-2.3 at a temperature above 200° C. and a pressure above 19 bar to provide a further hydrolysed cellulosic biomass and
e) fermenting the further hydrolysed cellulosic biomass with a microorganism to provide the hydrolysate liquid.

As a fourth configuration of the second aspect of the invention, there is provided a hydrolysate liquid obtainable by a process comprising the steps of:
a) providing cellulosic biomass;
b) impregnating the cellulosic biomass in a wood/liquid ratio of about from 1:1 to 1:7 to provide an impregnated cellulosic biomass;
c) hydrolysing the impregnated cellulosic biomass in a solution having a pH of 1.5-2.3 at a temperature of 160-200° C. and a pressure of 8-12 bar to provide a hydrolysed cellulosic biomass;
d) further hydrolysing the hydrolysed cellulosic biomass in a solution having a pH of 1.5-2.3 at a temperature above 200° C. and a pressure above 19 bar to provide a further hydrolysed cellulosic biomass;
e) fermenting the further hydrolysed cellulosic biomass using a microorganism to provide a fermentation broth and
f) distilling the fermentation broth to provide fermentation products and the hydrolysate liquid.

In an embodiment of the second aspect of the invention, the cellulosic biomass of step a) is lignocellulosic biomass.

As a third aspect of the present invention, there is provided the use of a hydrolysate liquid derived from a cellulosic biomass as an anti-corrosive agent.

An anti-corrosive agent refers to a product capable of decreasing the rate and/or the extent of corrosion. The third aspect of the disclosure is based on the inventor's insight that a hydrolysate liquid derived from cellulosic biomass is a corrosion inhibitor. In one embodiment of the second aspect, the anti-corrosive agent is capable of preventing corrosion of a material comprising iron. Material comprising iron may be sensitive to corrosion and is widely used in several industrial processes. The use of a hydrolysate liquid as an anti-corrosive agent capable of preventing corrosion of a material comprising iron may have several industrial applications. In one embodiment of the fourth aspect, the anti-corrosive agent is capable of preventing corrosion of a material comprising iron in an acid environment. Materials in acid environments may suffer from a high degree of corrosion.

As a fourth aspect of the present invention, there is provided the use of a hydrolysate liquid according the second aspect as an anti-corrosive agent. In an embodiment of the fourth aspect, the anti-corrosive agent is capable of preventing corrosion of a material comprising iron. In a further embodiment of the fourth aspect, the anti-corrosive agent is capable of preventing corrosion of a material comprising iron in an acid environment.

In an embodiment of the fourth aspect, the anti-corrosive agent is used in a process of pretreatment of cellulosic biomass. In another embodiment of this aspect, the pretreatment of the cellulosic biomass is the impregnation of the cellulosic biomass according step b) of the second aspect. The different terms used in the fourth aspect of the invention are referred to as described in the present disclosure herein above.

The anti-corrosive agent of the third or fourth aspect may also be a concentrate and/or filtrate of the hydrolysate liquid. In some embodiments, the hydrolysate or the concentrate thereof is mixed or reacted with one or more further substance(s) to facilitate its use as an anti-corrosive agent.

In embodiments of the third and fourth aspect, the hydrolysate liquid may contain no or substantially no solid residues of lignin. “Substantially no solid residues of lignin” may for example be a concentration of solid lignin in the hydrolyzate liquid of less than 5 g/L, such as less than 1 g/L.

As a fifth aspect of the present invention, there is provided a system for producing ethanol from cellulosic biomass comprising:
a) a pretreatment reactor for impregnating cellulosic biomass, connected to
b) at least one hydrolysis reactor for hydrolysing impregnated cellulosic biomass, further connected to
c) a fermentation apparatus for fermenting saccharides extracted from the cellulosic biomass in the at least one hydrolysis reactor to ethanol, further connected to
d) a distillation apparatus for separating ethanol from the cellulosic fermentation broth, further connected to
e) recirculation means for recirculating at least part of the cellulosic stillage obtained in the distillation apparatus to the pretreatment reactor,
wherein the fermentation apparatus of step c) and the distillation apparatus of step d) may be the same or different.

The activities of impregnating cellulosic biomass, hydrolysing impregnated cellulosic biomass, fermenting saccharides and separating ethanol from the cellulosic fermentation broth may be performed as described in the disclosure herein above. A pretreatment reactor and/or a hydrolysis reactor may be a vessel or a container. A distillation apparatus may be a distillation column. Stillage refers to the liquid effluent remaining after hydrolysis, fermentation and distillation of cellulosic biomass. Thus, the stillage may originate from a hydrolysate derived from cellulosic biomass. As an example, the stillage may be the liquid effluent obtained after pretreatment, at least one hydrolysis, fermentation and distillation of cellulosic biomass.

A system for producing ethanol from cellulosic biomass having recirculation means for recirculating the cellulosic stillage thus facilitates the use of the cellulosic stillage to inhibit corrosion in the pretreatment reactor. The recirculation means may be pipes connecting the distillation container with the pretreatment reactor. The recirculation may be adapted so that the at least part of the cellulosic stillage is mixed with a liquid before being recirculated to the pretreatment reactor. If the pretreatment involves impregnation, the at least part of the cellulosic stillage may be mixed with an acid solution before being recirculated to the pretreatment reactor. Further, the at least part of the cellulosic stillage may be mixed with a gas, such as SO₂-gas, before being recirculated to the pretreatment reactor. The recirculation means may also be connected to other apparatuses or containers used in the system for producing ethanol, so that the cellulosic stillage may be recirculated and used for inhibiting corrosion in more than one container or apparatus used in the system.

In one embodiment of the fifth aspect, the pretreatment reactor and/or the at least one hydrolysis reactor comprise iron in an amount of 50% or more by weight.

In one embodiment, the pretreatment reactor and/or the at least one hydrolysis reactor comprise iron in an amount of 50-75% by weight, chromium in an amount of 10.5-30% by weight, nickel in an amount of 2.5-29% by weight and molybdenum in an amount of 0-7% by weight.

In another embodiment of the fifth aspect, the pretreatment reactor and/or the at least one hydrolysis reactor comprise iron in an amount of 55-70% by weight, chromium in an amount of 15-26% by weight, nickel in an amount of 5-10% by weight and molybdenum in an amount of 1.5-5.5% by weight.

In a further embodiment, the pretreatment reactor and/or the at least one hydrolysis reactor are made of stainless steel.

In another embodiment of the fifth aspect the stainless steel is ferrite-austenitic stainless steel.

In one embodiment, the stainless steel is of the grade EN 1.4462 or EN 1.4410.

The terms and definitions used in the fifth aspect of the invention are the same as referred to in the other aspects of the invention herein above.

As a sixth aspect of the present invention, there is provided an apparatus for impregnation of a cellulosic biomass under the usage of an acidic impregnation fluid of pH 0.5-5.5 comprising recycled and an optionally purified stillage, to obtain a impregnated cellulosic biomass in a process for production of ethanol liquid from the cellulosic biomass, the process further comprising hydrolysis of the impregnated biomass to obtain a hydrolysate, fermentation of the hydrolysate to obtain a fermentation broth, distillation of the fermented hydrolysate to provide the ethanol stream and the stillage, and optionally, purification of the stillage, wherein the parts of the apparatus that contain the impregnation fluid consist of a material comprising iron in an amount of at least 50% by weight, chromium in an amount of 10.5-30% by weight, nickel in an amount of 2.5-29% by weight and molybdenum in an amount of 0-7% by weight.

The terms and definitions used in the sixth aspect of the invention are the same as referred to in the other aspects of the invention herein above. Also, the skilled person understands that embodiments, especially the process steps and conditions, of the sixth aspect may be specified according to the embodiments of the first to fifth aspect.

In one embodiment, the apparatus further comprises a mixer adapted to mix the optionally purified stillage with an acid to produce the acidic impregnation fluid and an inlet in connection with the mixer adapted to receive the acidic impregnation fluid.

As a seventh aspect of the invention, there is provided the use of a material for the containment of an acidic impregnation fluid comprising a stillage from a distillation of a cellulosic fermentation broth derived from a cellulosic biomass, the impregnation fluid having a pH of 0.5-5.5, by means of which impregnation fluid a cellulosic biomass is impregnated at a temperature of 20-150° C., a pressure of 1-5 bar and a time of 1-120 minutes, wherein the material comprises iron in an amount of at least 50% by weight, chromium in an amount of 10.5-30% by weight, nickel in an amount of 2.5-29% by weight and molybdenum in an amount of 0-7% by weight.

The terms and definitions used in the seventh aspect of the invention are the same as referred to in the other aspects of the invention herein above. Also, the skilled person understands that embodiments, especially the process steps and conditions, of the seventh aspect may be specified according to the embodiments of the first to fifth aspect.

### BRIEF DESCRIPTION OF THE DRAWINGS

FIG. 1 shows OCP vs. time for the duplex 2205 surface using the 3 wt. % control NaCl solution and the GHA and GHB hydrolysates (open squares=Control NaCl, filled squares=GHA 150 ml/L, open circles=GHA 30 ml/L, filled circles=GHB 150 ml/L, open triangles=GHB 30 ml/L).

FIG. 2 shows OCP vs. time for the duplex SAF 2507 surface using the 3 wt. % control NaCl solution and the GHA and GHB hydrolysates (open squares=Control NaCl, filled squares=GHA 150 ml/L, open circles=GHA 30 ml/L, filled circles=GHB 150 ml/L, open triangles=GHB 30 ml/L).

FIG. 3 shows Nyquist plots (FIG. 3A) and Bode plots (FIG. 3B) for the duplex 2205 surface exposed to two samples of 3 wt. % NaCl without addition of any hydrolysate (open squares=Control NaCl 1 hour (1), filled squares=Control NaCl 1 day (1), open circles=Control NaCl 1 hour (2), filled circles=Control NaCl 1 day(2)).

FIG. 4 shows Nyquist plots (FIG. 4A) and Bode plots (FIG. 4B) for the duplex SAF 2507 surface exposed to two samples of 3 wt. % NaCl without addition of any hydrolysate (open squares=Control NaCl 1 hour (1), filled squares=Control NaCl 1 day (1), open circles=Control NaCl 1 hour (2), filled circles=Control NaCl 1 day(2)).

FIG. 5 shows Nyquist plots (FIG. 5A) and Bode plots (FIG. 5B) for the duplex 2205 surface exposed to 30 ml/L GHA (open squares=GHA 30 ml/L 1 hour, filled squares=GHA 30 ml/L 8 hours, open circles=GHA 30 ml/L 1 day, filled circles=GHA 30 ml/L 3 days).

FIG. 6 shows Nyquist plots (FIG. 6A) and Bode plots (FIG. 6B) for the duplex 2205 surface exposed to 150 ml/L GHA (open squares=GHA 150 ml/L 1 hour, filled squares=GHA 150 ml/L 8 hours, open circles=GHA 150 ml/L 1 day, filled circles=GHA 150 ml/L 3 days).

FIG. 7 shows Nyquist plots (FIG. 7A) and Bode plots (FIG. 7B) for the duplex SAF 2507 surface exposed to 30 ml/L GHA (open squares=GHA 30 ml/L 1 hour, filled squares=GHA 30 ml/L 8 hours, open circles=GHA 30 ml/L 1 day, filled circles=GHA 30 ml/L 3 days).

FIG. 8 shows Nyquist plots (FIG. 8A) and Bode plots (FIG. 8B) for the duplex SAF 2507 surface exposed to 150 ml/L GHA (open squares=GHA 150 ml/L 1 hour, filled squares=GHA 150 ml/L 8 hours, open circles=GHA 150 ml/L 1 day, filled circles=GHA 150 ml/L 3 days).

FIG. 9 shows Nyquist plots (FIG. 9A) and Bode plots (FIG. 9B) for the duplex 2205 surface exposed to 30 ml/L GHB (open squares=GHB 30 ml/L 1 hour, filled squares=GHB 30 ml/L 8 hours, open circles=GHB 30 ml/L 1 day, filled circles=GHB 30 ml/L 3 days).

FIG. 10 shows Nyquist plots (FIG. 10A) and Bode plots (FIG. 10B) for the duplex 2205 surface exposed to 150 ml/L GHB (open squares=GHB 150 ml/L 1 hour, filled squares=GHB 150 ml/L 8 hours, open circles=GHB 150 ml/L 1 day, filled circles=GHB 150 ml/L 3 days).

FIG. 11 shows Nyquist plots (FIG. 11A) and Bode plots (FIG. 11B) for the duplex SAF 2507 surface exposed to 30 ml/L GHB (open squares=GHB 30 ml/L 1 hour, filled squares=GHB 30 ml/L 8 hours, open circles=GHB 30 ml/L 1 day, filled circles=GHB 30 ml/L 3 days).

FIG. 12 shows Nyquist plots (FIG. 12A) and Bode plots (FIG. 12B) for the duplex SAF 2507 surface exposed to 150 ml/L GHB (open squares=GHB 150 ml/L 1 hour, filled squares=GHB 150 ml/L 8 hours, open circles=GHB 150 ml/L 1 day, filled circles=GHB 150 ml/L 3 days).

FIG. 13shows OCP vs. time for the duplex 2205 surface using the 3 wt. % control NaCl solution and the GHC hydrolysate. For comparison, the GHA and GHB hydrolysates are included in the plot (open squares=Control NaCl, filled squares=GHA 30 ml/L, open circles=GHB 30 ml/L, filled circles=GHC 30 ml/L).

FIG. 14 shows Nyquist plots (FIG. 14A) and Bode plots (FIG. 14B) for the duplex 2205 surface exposed to 30 ml/L GHC (open squares=GHC 30 ml/L 1 hour, filled squares=GHC 30 ml/L 8 hours, open circles=GHC 30 ml/L 1 day, filled circles=GHC 30 ml/L 3 days).

### EXAMPLES

The following non-limiting examples will further illustrate the present invention.

### Example 1

### Corrosion Inhibition of Spruce Hydrolysate Obtained after Acid Hydrolysis

### Materials

Hydrolysate was prepared from spruce chippings having a humidity of 50%. The spruce chippings were continuously fed to a hydrolysis reactor at a speed of 50 kg/h, together with 0.8% H₂SO₄ at a speed of 30 L/hour. The environmental conditions inside the reactor were a temperature of 175° C. and a pressure of 9 bar and the retention time in the reactor for the biomass was 5 min. Formed hydrolysate from the biomass was continuously squeezed out from the reactor. The preparation procedure was performed in duplicate and the prepared hydrolysates were denoted GHA and GHB, respectively. The hydrolysates were highly acidic, having a pH of about 1.8. The prepared GHA and GHB were respectively diluted in 3 wt. % NaCl solution to form two different concentrations; 30 ml/L, i.e. 30 ml of GHA was added into 3 wt. % NaCl solution to make a 1 L test solution, and 150 ml/L, i.e. 150 ml of GHA was added into 3 wt. % NaCl solution to make a 1 L test solution. As a reference or a control sample, a solution containing only 3 wt. % NaCl and having a pH of about 7 was used.

Tested surfaces were coupons of two duplex stainless steel grades, 2205 and SAF 2507, provided by KIMAB (Stockholm, Sweden). The duplex 2205 surface was of grade EN 1.4462 and the SAF 2507 surface was of grade EN 1.4410. The sample surfaces were ground successively using SiC paper to 1200 grit and cleaned with ethanol and acetone prior to the corrosion test.

### Corrosion Tests

The corrosion was tested using electrochemical measurements including Open-circuit potential (OCP) and Electrochemical impedance spectroscopy (EIS). The electrochemical cells used consisted of three electrodes, a saturated Ag/AgCl reference electrode, a Pt mesh counter electrode, and the sample surface as the working electrode. The exposed sample surface area was 1 cm³ in all cases. Two sets of instruments, a Solartron 1286 electrochemical interface coupled with a Solartron 1255 frequency response analyzer, and a Solartron 1287 electrochemical interface coupled with a Solartron 1250 frequency response analyzer, were used for the measurements. The instruments were controlled using a computer with the CorrWare and Zplot software.

### Results OCP-measurements

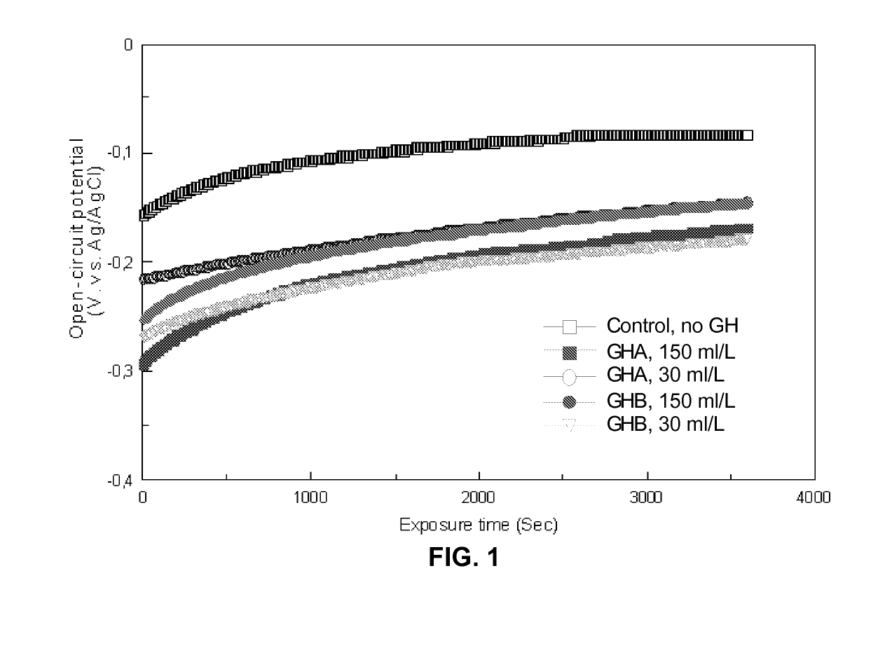

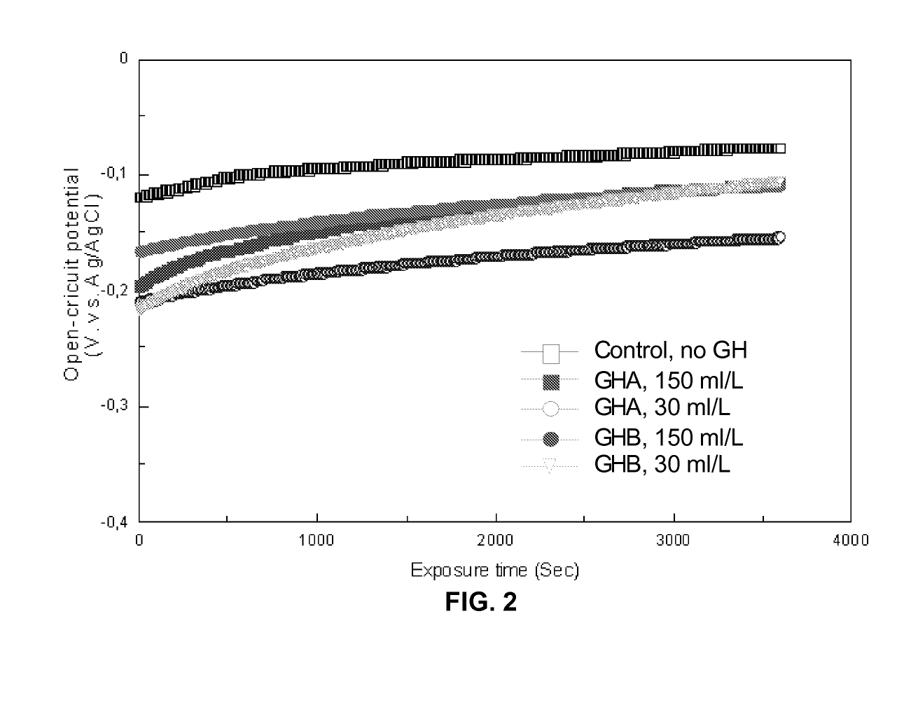

The results from the OCP-measurements using the control NaCl-solution of pH 7 and different concentrations of the GHA and GHB solutions, 30 ml/L and 150 ml/L, respectively, are displayed in FIG. 1 and FIG. 2. The OCP data clearly showed that the addition of GHA and GHB led to a lower OCP on both the duplex stainless steel 2205 surface (FIG. 1) and the duplex SAF 2507 surface (FIG. 2) for both concentrations of GHA and GHB. The decrease in OCP with addition of the GHA and GHB compared to the NaCl solution is surprising, since the hydrolysates are very acidic compared to the NaCl solution of pH 7. The decrease of the OCP indicated a cathodic type of inhibition effect of the GHA and GHB, especially on the duplex 2205 surface.

### Results EIS-Measurements Using Control NaCl Solutions

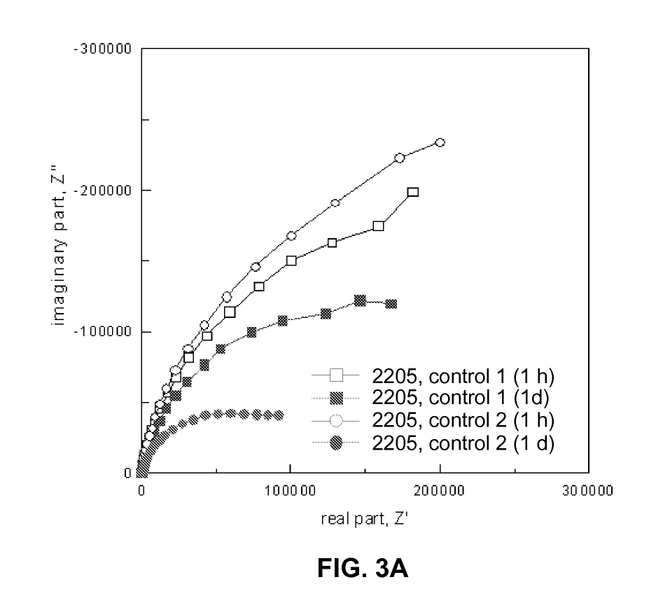

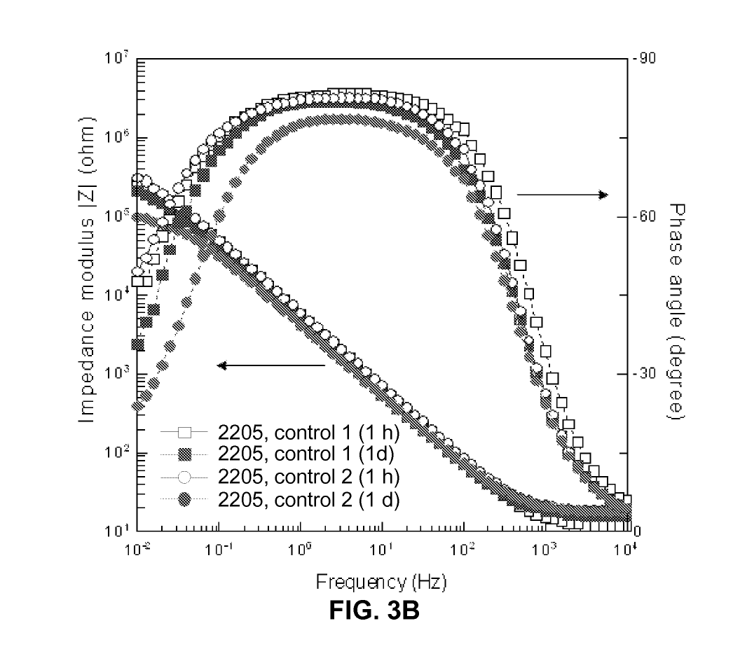

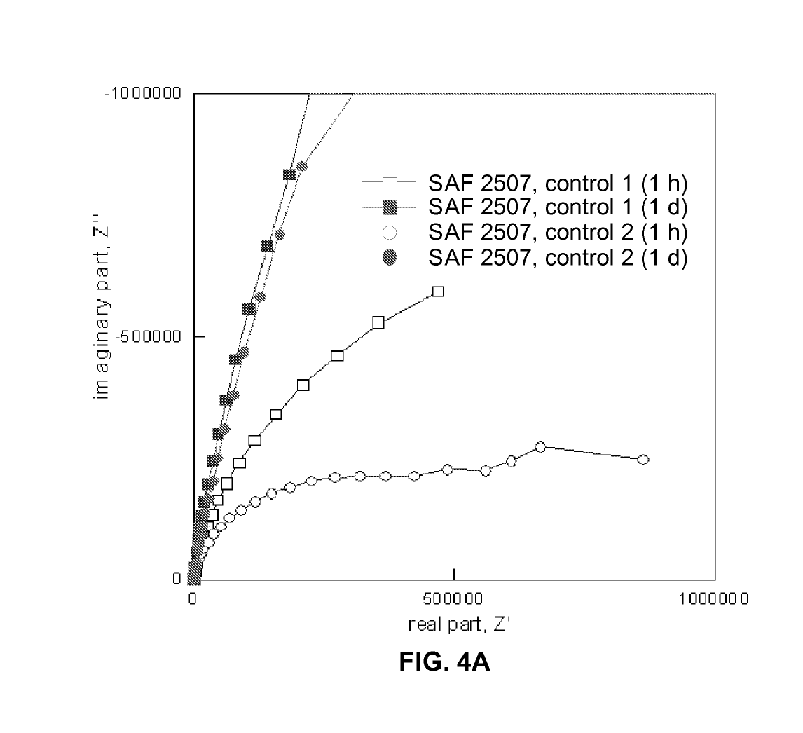

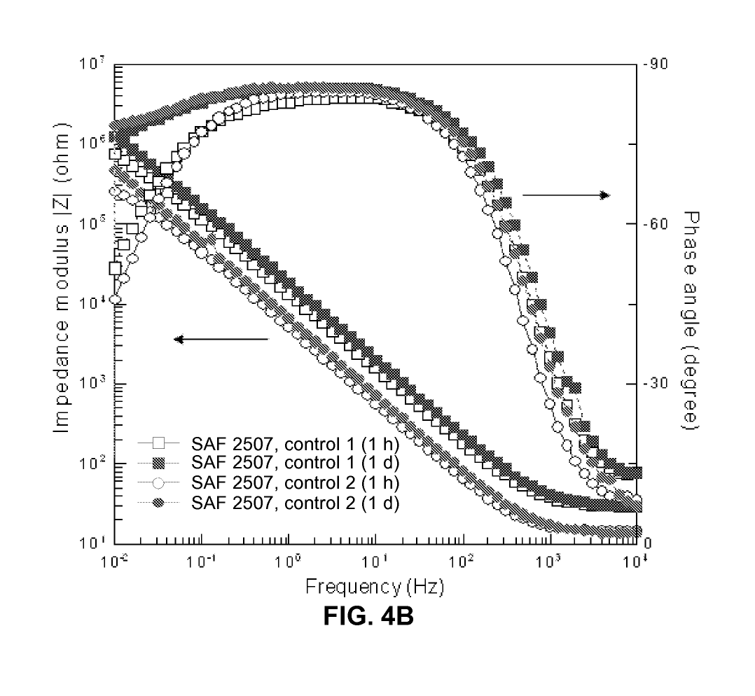

The results from the EIS-measurements using the control NaCl-solution of pH 7 and duplex stainless steel 2205 surface are displayed in FIG. 3 and the results using the control NaCl solution and the duplex SAF 2507 surface are displayed in FIG. 4. Spectra were obtained after 1 hour and after 1 day. With only the control NaCl solution, the EIS spectra on the duplex stainless steel 2205 surface exhibited one time-constant feature (near-symmetric angle curve in the Bode plots). This indicated that no adsorbed film was present on the surface (FIG. 3B). The polarization resistance Rₚ, which is a measure of the corrosion resistance, was obtained from quantitative analysis of the spectra based on spectra fitting using a simple equivalent circuit. It was evident from the Nyquist plot in FIG. 3A that the polarization resistance decreased with the exposure time. This indicated that the duplex 2205 surface suffered from corrosion in the control NaCl solution. On the duplex SAF 2507 surface, the behavior was somewhat different (FIG. 4). The polarization resistance was about one order of magnitude higher compared to the duplex 2205 surface. Moreover, the polarization resistance increased with exposure time, which suggested an high corrosion resistance of the duplex SAF 2507 surface in the solution. Thus, of the two tested surfaces in the control NaCl solution, the duplex 2205 surface suffered from the highest degree of corrosion.

### Results EIS-Measurements Using GHA and GHB

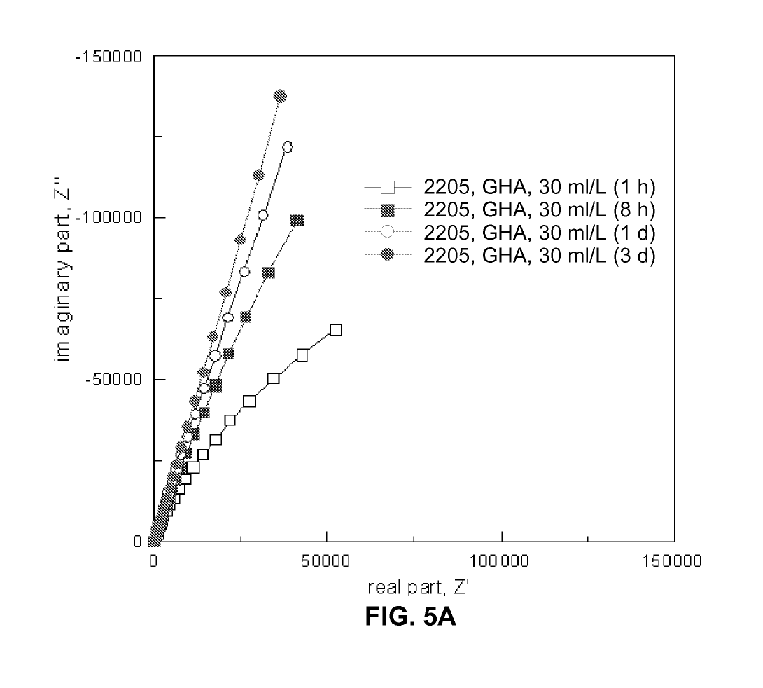

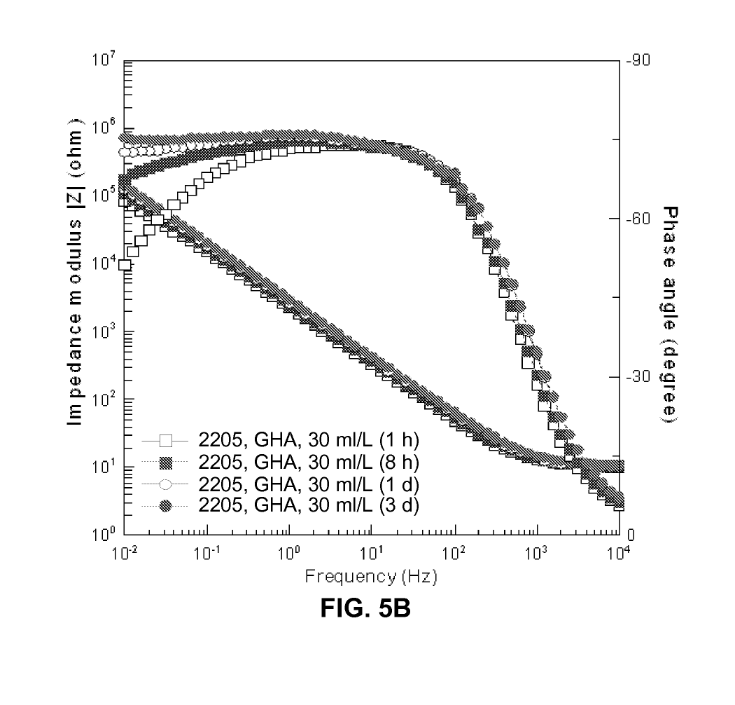

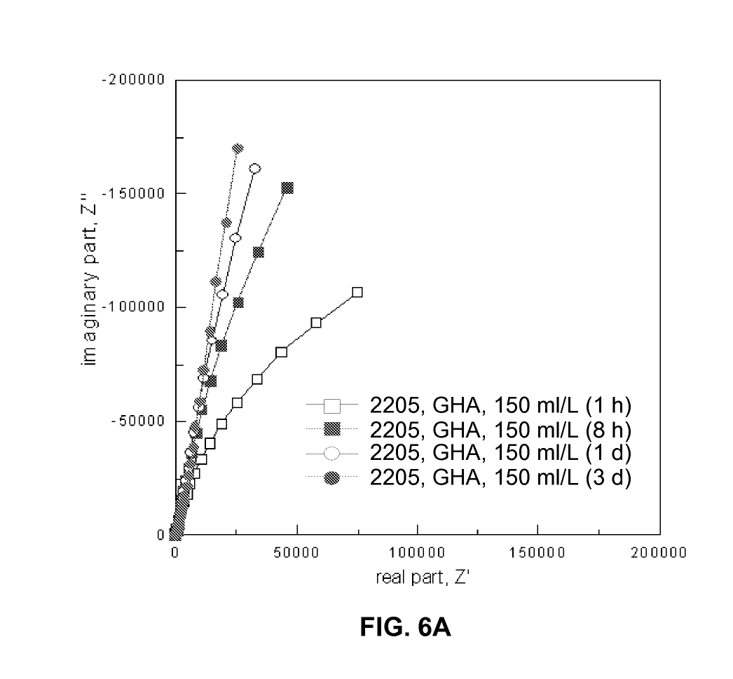

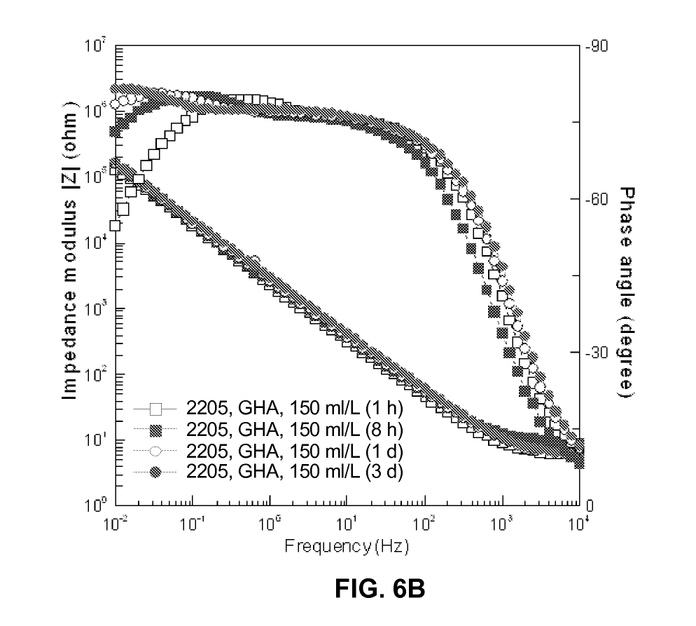

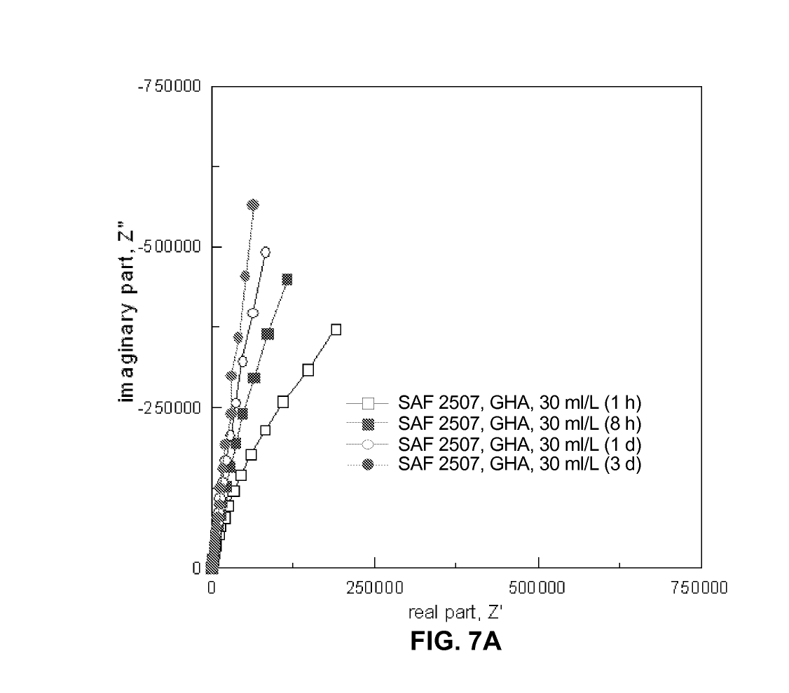

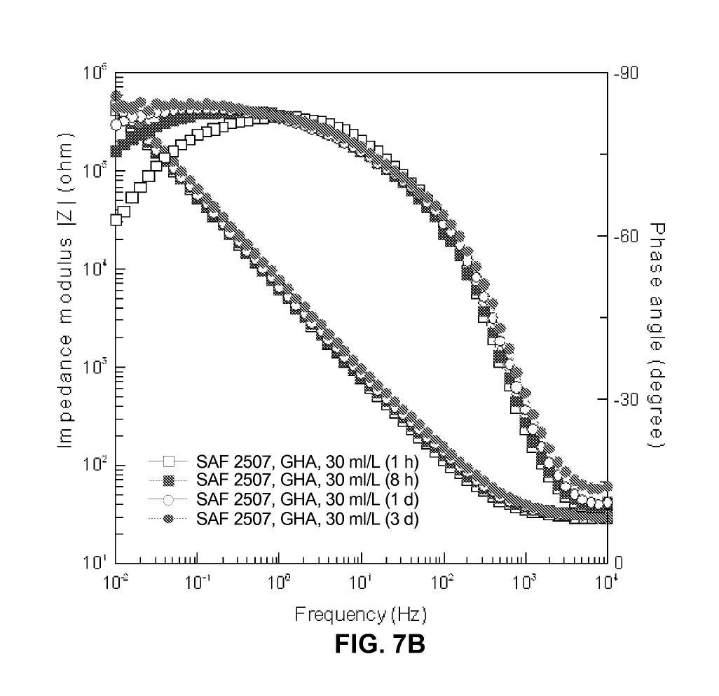

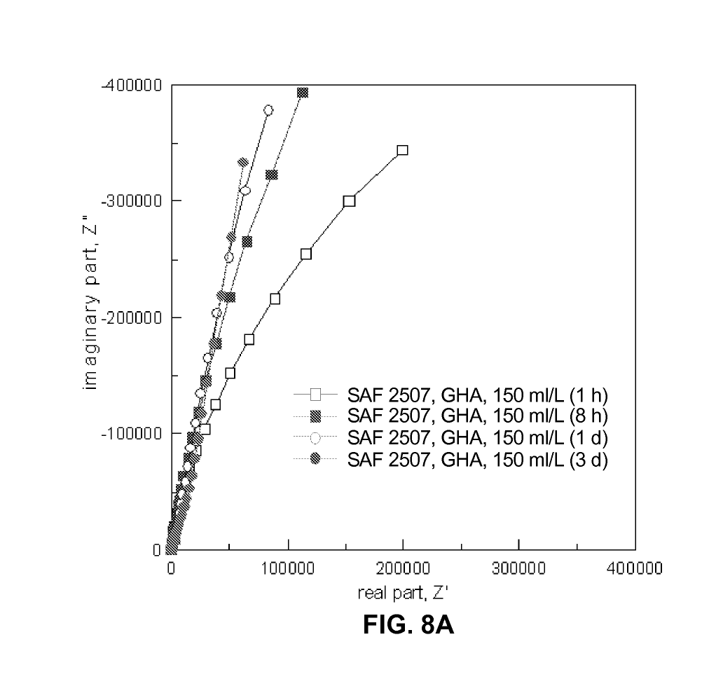

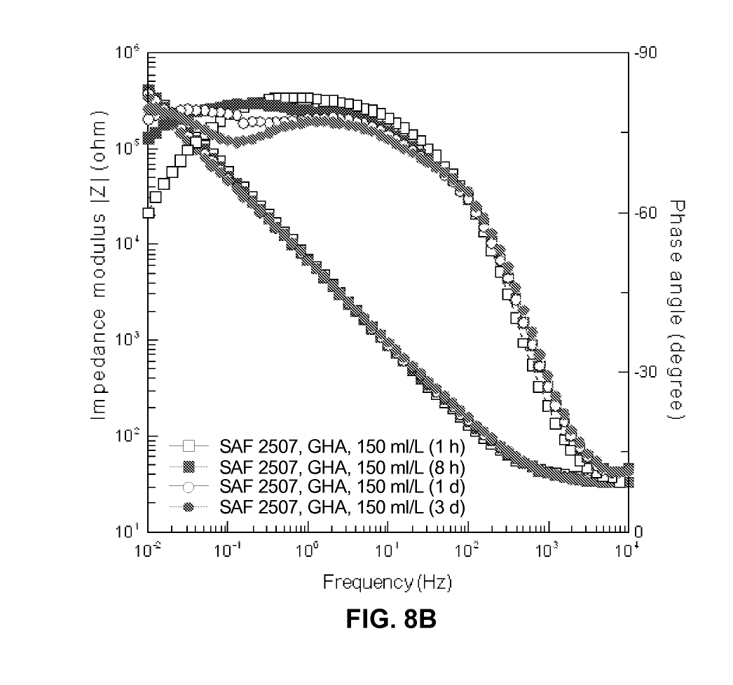

The results using different concentrations of the GHA and GHB solutions, 30 ml/L and 150 ml/L, respectively, on the two different surfaces are displayed in FIGS. 5-12. The spectra using the duplex 2205 surface with two different concentrations of GHA (FIGS. 5 and 6) revealed that, with the addition of GHA, the polarization resistance increased with exposure time, i.e. the opposite compared to the control NaCl sample. Thus, the results indicated a corrosion inhibiting effect of the GHA. Using 150 ml/L GHA (FIG. 6), the phase angle data suggested an additional time constant. This was evident in the Bode plot (FIG. 6B), which revealed double peaks, i.e. a low frequency response associated with an adsorbed layer on the surface. The EIS spectra for the duplex SAF 2507 surface with a low level (30 ml/L) and a high level (150 ml/L) of GHA are displayed in FIGS. 7 and 8. For both concentrations, the polarization resistance increased with exposure time to a very high level. Moreover, with a high concentration of GHA, the low frequency data suggested an adsorbed layer on the surface, which indicated a certain corrosion inhibiting effect of GHA. Hence, the EIS data revealed a corrosion inhibiting effect of the GHA, especially on the duplex 2205 surface, and an adsorbed layer of GHA on both surfaces.

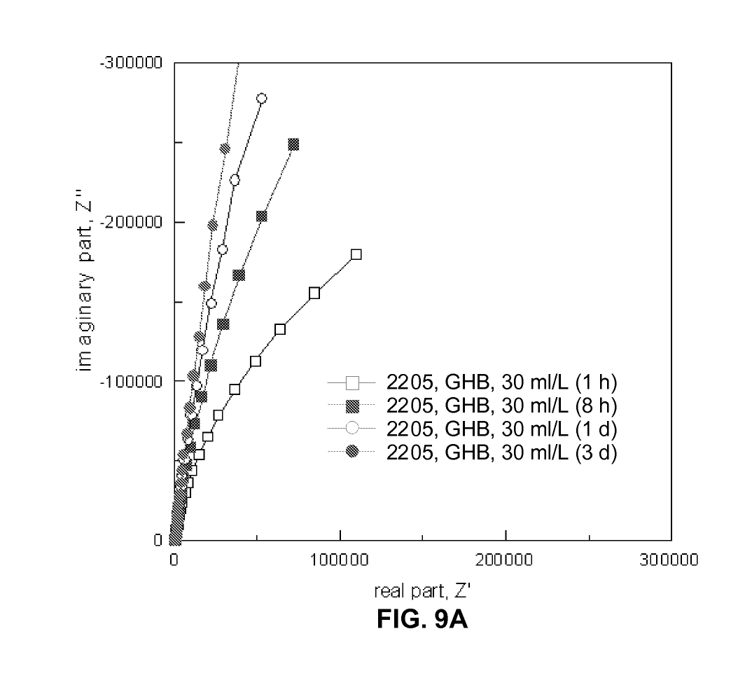

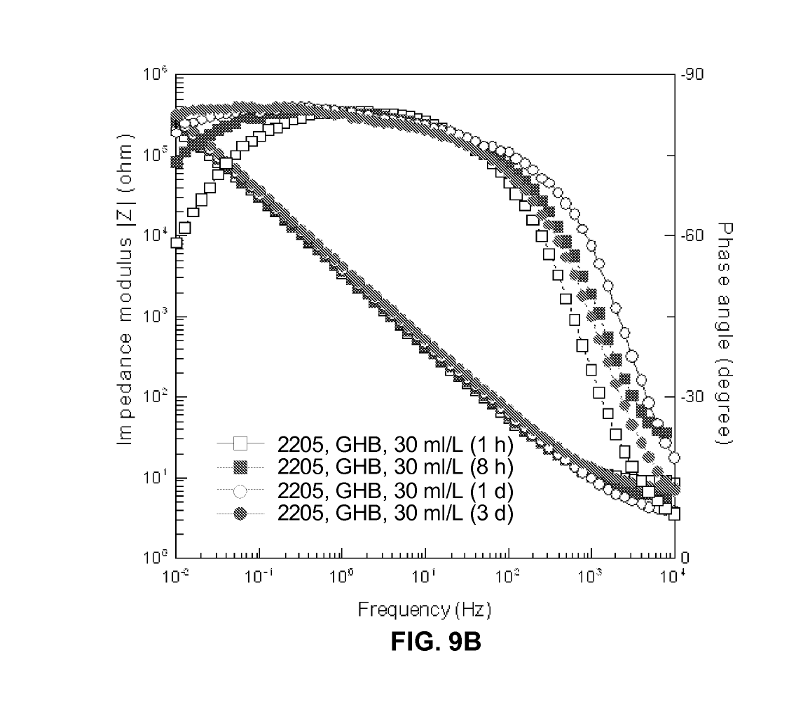

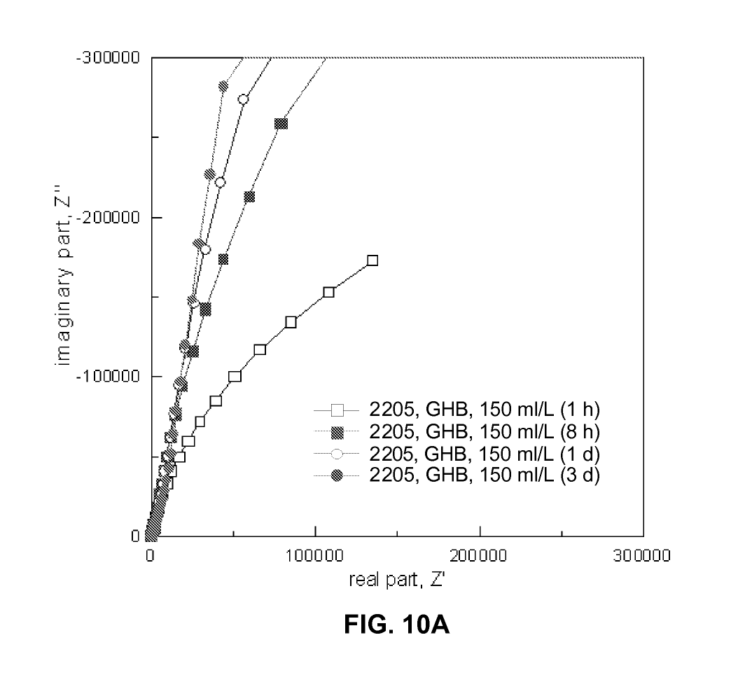

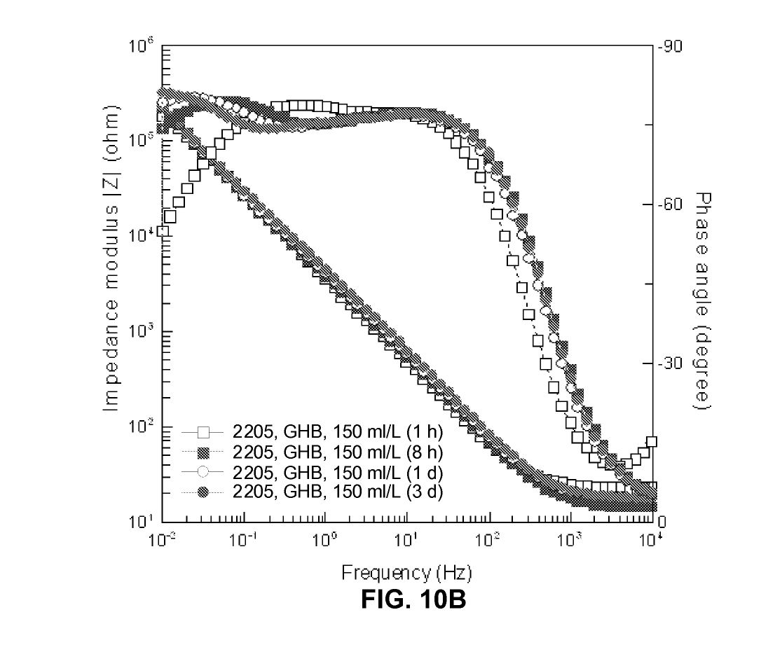

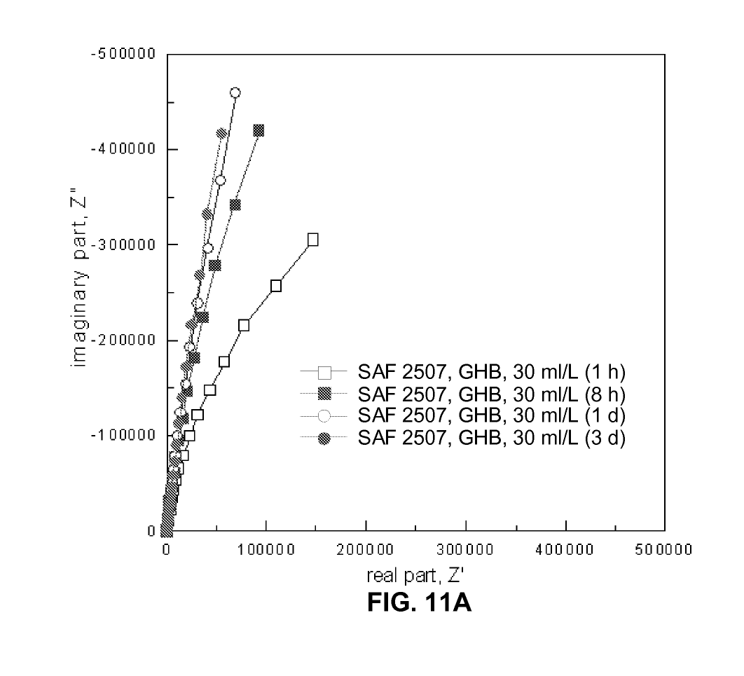

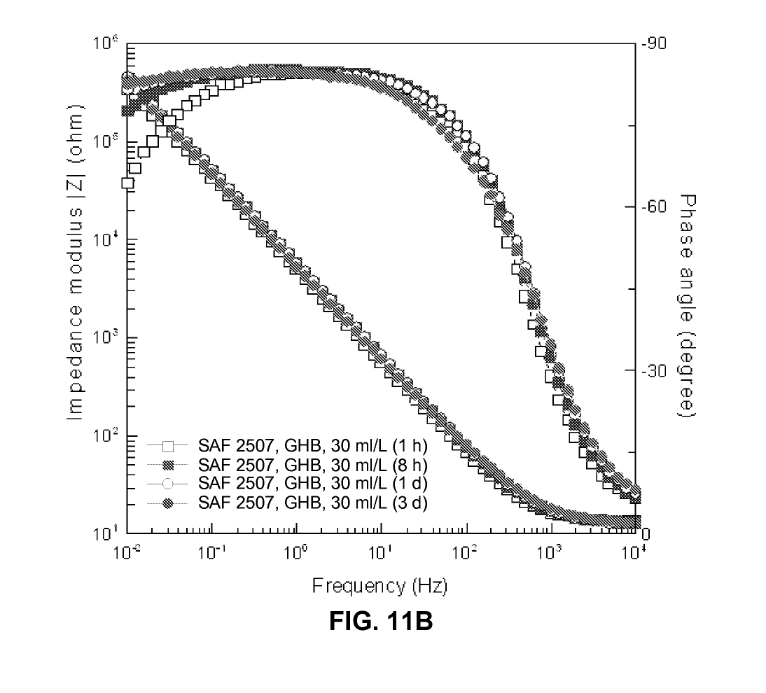

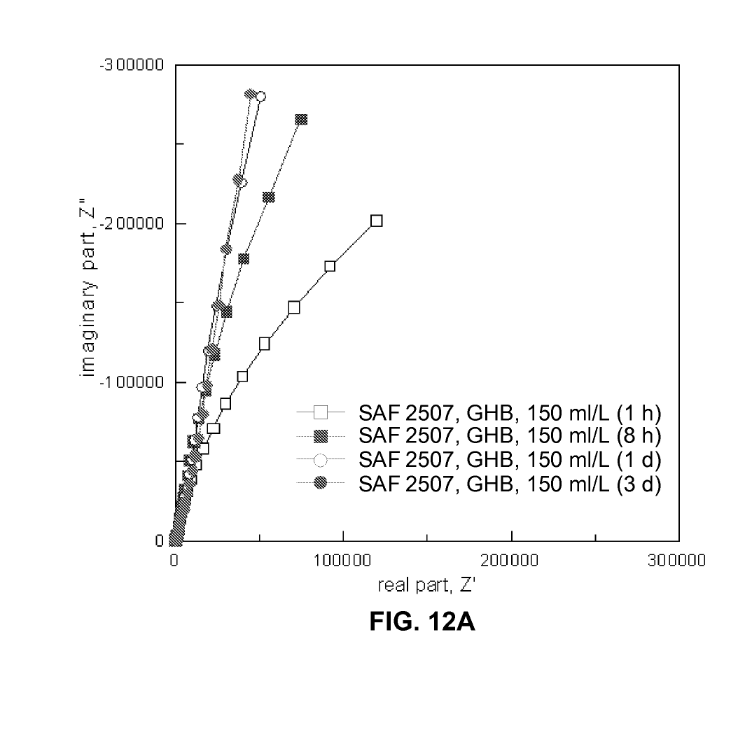

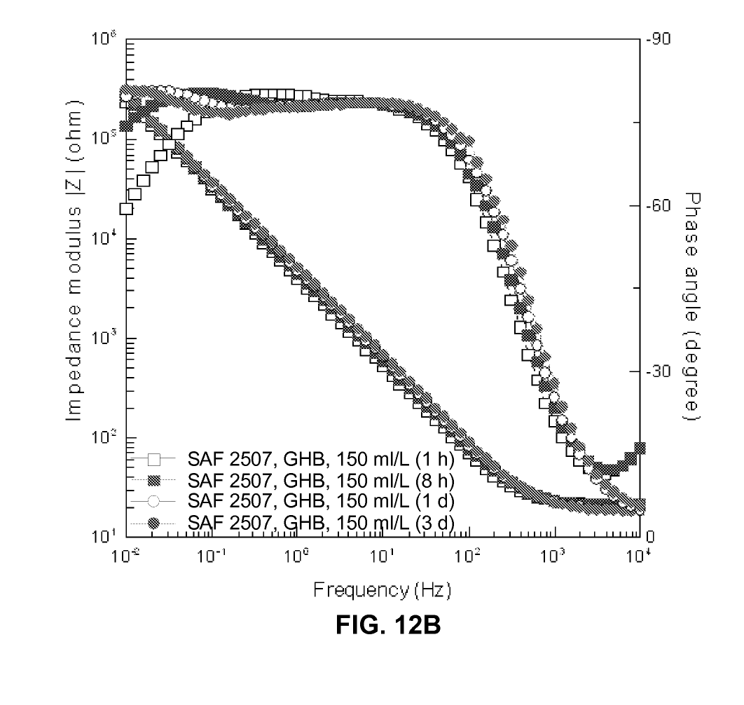

The spectra using the duplex 2205 surface with two different concentrations of GHB (FIGS. 9 and 10) revealed that, with the addition of GHB, the polarization resistance increased with exposure time, i.e. the opposite compared to the control NaCl sample. Thus, the results, similar as to when using GHA, indicated a corrosion inhibiting effect of the GHB. Using 150 ml/L GHB (FIG. 10), the phase angle data suggested an additional time constant. This was evident in the Bode plot (FIG. 10B), which revealed double peaks, i.e. a low frequency response associated with an adsorbed layer on the surface. The EIS spectra for the duplex SAF 2507 surface with a low level (30 ml/L) and a high level (150 ml/L) of GHB are displayed in FIGS. 11 and 12, respectively. For both concentrations of hydrolysate, the polarization resistance increased with exposure time to a very high level. Moreover, with a high concentration of GHB, the low frequency data suggested an adsorbed layer on the surface, which indicated a certain corrosion inhibiting effect of GHB. Hence, as with GHA, the EIS data revealed a corrosion inhibiting effect of the GHB, especially on the duplex 2205 surface, and an adsorbed layer of GHB on both surfaces.

To summarize, the EIS data showed an inhibiting effect on both types of stainless steel surfaces when using both the GHA and the GHB hydrolysates.

Compared to the NaCl reference solution of pH 7, the hydrolysates showed a superior corrosion inhibiting effect, even though the hydrolysates were much more acidic than the NaCl solution.

### Example 2

### Corrosion Inhibition of Spruce Stillage Obtained after Acid Hydrolysis Followed by Fermentation and Distillation

### Materials

Filtered stillage was prepared from spruce chippings having a humidity of 50%. The spruce chippings were continuously fed at a speed of 50 kg/h dry matter to an impregnation step where the chips were impregnated with 2 kg/h sulphur dioxide, SO₂, for 20 minutes. From the impregnation vessel the spruce chippings were continuously fed into the first hydrolysis reactor. The environmental conditions inside the first reactor were a temperature of 175° C. and a pressure of 9 bar and the retention time in the reactor for the biomass was 5 min. Formed hydrolysate from the biomass was continuously squeezed out from the first reactor. The solid residue from the first reactor step was continuously fed into the second reactor step. The environmental conditions inside the second reactor were a temperature of 210° C. and a pressure of 22 bar and the retention time in the reactor for the biomass was 5 min. From the second reactor step the formed slurry was combined with the hydrolysate from the first reactor stage. The combined product from the reactor stages were filtered in a membrane filter press. The liquid phase, i.e. the sugar rich hydrolysate, was pH adjusted to pH 5 by sodium hydroxide, NaOH, and pumped to the fermentation vessel. Baker's yeast, Saccharomyces cerevisiae was added to the fermentation vessel in order to ferment the sugars to ethanol. After the fermentation was completed the fermentation broth was pumped to the distillation column where the ethanol was separated from the stillage. A sample was taken from the distillation stillage and filtered through a standard laboratory filter paper in order to remove solid residues. The filtered stillage had a pH of 4.5. The filtered stillage sample was given the name GHC.

The prepared GHC was diluted in 3 wt. % NaCl solution to form a concentration of 30 ml/L, i.e. 30 ml of GHC was added into 3 wt. % NaCl solution to make a 1 L test solution. As a reference or a control sample, a solution containing only 3 wt. % NaCl and having a pH of about 7 was used.

Tested surface was coupons of a stainless steel surface of duplex 2205 grade, provided by KIMAB (Stockholm, Sweden). The duplex 2205 surface was of grade EN 1.4462 and the SAF 2507 surface was of grade EN 1.4410. The sample surfaces were ground successively using SiC paper to 1200 grit and cleaned with ethanol and acetone prior to the corrosion test.

### Corrosion Tests

The corrosion was tested using electrochemical measurements including Open-circuit potential (OCP) and Electrochemical impedance spectroscopy (EIS) as described in Example 1.

### Results OCP-Measurements

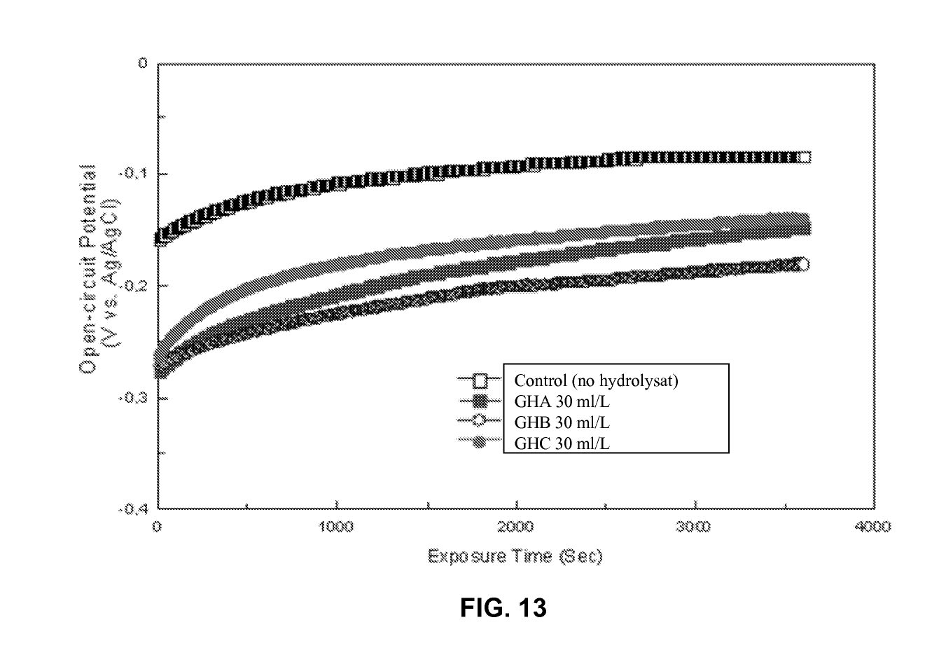

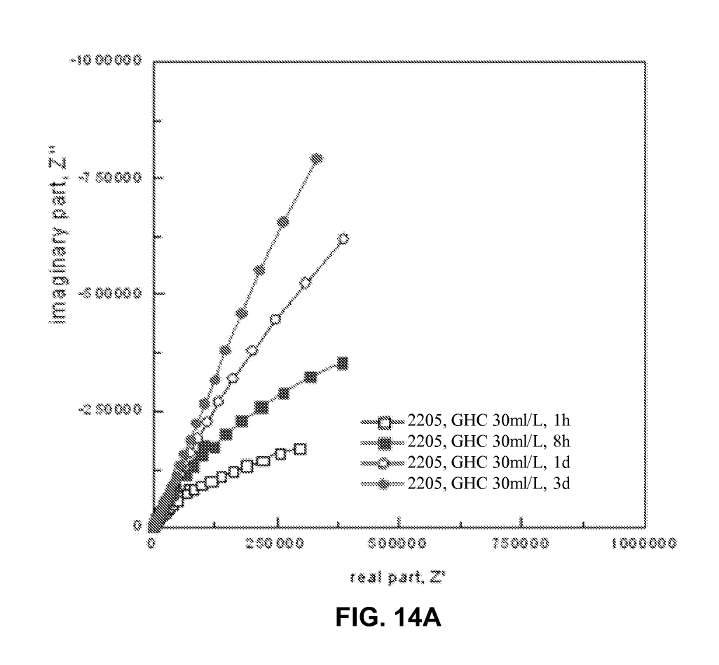

The results from the OCP-measurements using the control NaCl-solution of pH 7 and the GHC of 30 ml/L is displayed in FIG. 13. As a reference, the results using GHA and GHB are plotted in the same Figure. The OCP data clearly showed that the addition of GHC led to a lower OCP on the duplex stainless steel 2205 surface. The decrease in OCP with addition of the GHC hydrolysate compared to the NaCl solution is surprising. The decrease of the OCP indicated a cathodic type of inhibition effect of the GHC hydrolysate on the duplex 2205 surface.

### Results EIS-Measurements Using GHC

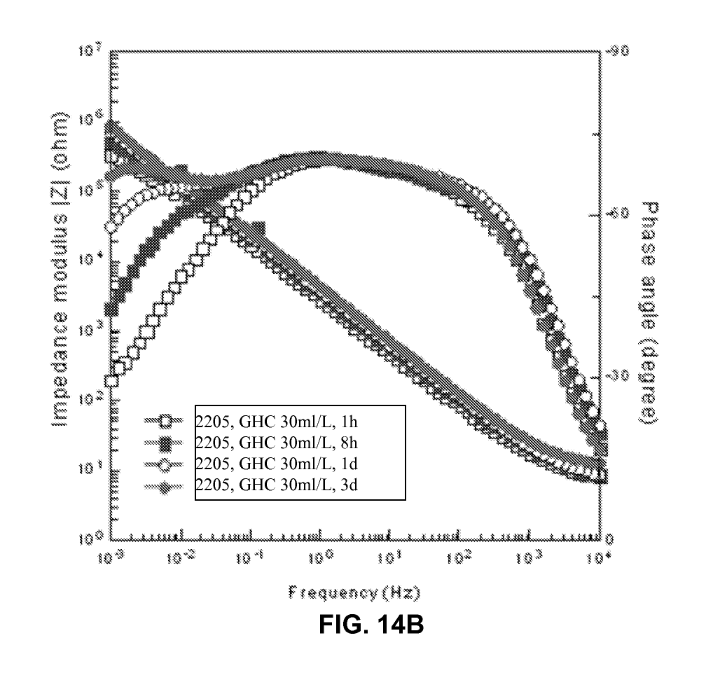

The results using 30 ml/L of GHC on the duplex 2205 surface are displayed in FIG. 14. The spectra revealed that the polarization resistance increases with exposure time when adding GHC, which is opposite to the control sample. The results clearly indicate an inhibiting effect of the GHC filtered stillage.

As can be seen in the Bode plot in FIG. 14B, the phase angle data at low frequencies indicated an additional time constant in the spectra, which suggested that an adsorbed film was formed on the metal surface. When using GHA and GHB, this behavior was only observed when a higher concentration (150 ml/L) of the hydrolysate was added into the solution, as seen in FIGS. 6 and 10. Moreover, as judged from the impedance modulus at low frequencies (corresponding the polarization resistance), GHC seemed to give a larger inhibition effect than the GHA and GHB hydrolysates.

Thus, this example clearly showed the inhibiting effect of a filtered stillage obtained after hydrolysis and fermentation of cellulose biomass.

1. A method for inhibiting corrosion comprising the steps of:
a) subjecting cellulosic biomass to hydrolysis to obtain a hydrolysate liquid;
b) optionally, subjecting the hydrolysate liquid from step a) to one or more of the steps of further hydrolysis, fermentation and/or distillation;
c) using the hydrolysate liquid from step a) or b) to inhibit corrosion.

2. The method according to claim 1, wherein step c) comprises adding said hydrolysate liquid in a process to inhibit corrosion of one or more apparatuses used in said process.

3. The method according to claim 2, wherein said hydrolysate liquid of step c) is added as at least part of a process fluid in said process, and said one or more apparatuses are in contact with said process fluid.

4. The method according to claim 3, wherein the pH of said process fluid is 0-6, such as 0.5-4.5.

5. The method according to claim 2, wherein said one or more apparatuses comprise iron.

6. The method according to claim 5, wherein said one or more apparatuses comprise iron in an amount of 50% or more by weight.

7. The method according to claim 6, wherein said one or more apparatuses comprise iron in an amount of 50-75% by weight, chromium in an amount of 10.5-30% by weight, nickel in an amount of 2.5-29% by weight and molybdenum in an amount of 0-7% by weight.

8. The method according to claim 7, wherein said one or more apparatuses comprise iron in an amount of 55-70% by weight, chromium in an amount of 15-26% by weight, nickel in an amount of 5-10% by weight and molybdenum in an amount of 1.5-5.5% by weight.

9. The method according to claim 2, wherein said one or more apparatuses are made of stainless steel.

10. The method according to claim 9, wherein said stainless steel is ferrite-austenitic stainless steel.

11. The method according to claim 10, wherein the stainless steel is of the grade EN 1.4462 or EN 1.4410.

12. The method according to claim 1, wherein the hydrolysate liquid of step c) is added in a step i), which is performed upstream of step a).

13. The method according to claim 12, wherein step i) comprises pretreatment of the cellulosic biomass.

14. The method according to claim 13, wherein said pretreatment is impregnation.

15. The method according to claim 1, wherein the cellulosic biomass is lignocellulosic biomass.

16. The method according to claim 1, wherein step a) and/or the further hydrolysis of step b) comprise subjecting said cellulosic biomass to an acid hydrolysis.

17. The method according to claim 16, wherein step a) and/or the further hydrolysis of step b) comprise subjecting said cellulosic biomass to an acid hydrolysing liquid to which a mineral acid, such as H₂SO₄, and/or SO₂ gas has been added.

18.-40. (canceled)

41. Method of preventing corrosion of a material, comprising the step of providing a hydrolysate liquid derived from a cellulosic biomass as an anti-corrosive agent.

42. Method according to claim 41, wherein said material comprises iron.

43. Method of preventing corrosion of a material comprising iron in an acidic environment, comprising the step of providing a hydrolysate liquid derived from a cellulosic biomass as an anti-corrosive agent.

44. Method according to claim 41, wherein said hydrolysate liquid is obtainable by a process comprising the steps of:
a) providing cellulosic biomass;
b) impregnating the cellulosic biomass in a wood/liquid ratio of about from 1:1 to 1:7 to provide an impregnated cellulosic biomass and
c) hydrolysing the impregnated cellulosic biomass in a solution having a pH of 1.5-2.3 at a t temperature of 160-200° C. and a pressure of 8-12 bar to provide said hydrolysate liquid or a pre-hydrolysed cellulosic biomass; and optionally
d) further hydrolysing the pre-hydrolysed cellulosic biomass in a solution having a pH of 1.5-2.3 at a temperature above 200° C. and a pressure above 19 bar to provide said hydrolysate liquid or a hydrolysed cellulosic biomass; and optionally
e) fermenting the hydrolysed cellulosic biomass using a microorganism to provide said hydrolysate liquid or a fermentation broth; and optionally
f) distilling the fermentation broth to provide fermentation products and said hydrolysate liquid.

45. A system for producing ethanol from cellulosic biomass comprising:
a) a pretreatment reactor for impregnating cellulosic biomass, connected to
b) at least one hydrolysis reactor for hydrolysing impregnated cellulosic biomass, further connected to
c) a fermentation apparatus for fermenting saccharides extracted from said cellulosic biomass in said at least one hydrolysis reactor to ethanol, further connected to
d) a distillation apparatus for separating ethanol from the cellulosic fermentation broth, further connected to
e) recirculation means for recirculating at least part of the cellulosic stillage obtained in the distillation apparatus to the pretreatment reactor,
wherein the fermentation apparatus of step c) and the distillation apparatus of step d) may be the same or different, wherein said pretreatment reactor and/or said at least one hydrolysis reactor comprise iron in an amount of 50-75% by weight, chromium in an amount of 10.5-30% by weight, nickel in an amount of 2.5-29% by weight and molybdenum in an amount of 0-7% by weight.

46. System according to claim 45, wherein said pretreatment reactor and/or said at least one hydrolysis reactor comprise iron in an amount of 55-70% by weight, chromium in an amount of 15-26% by weight, nickel in an amount of 5-10% by weight and molybdenum in an amount of 1.5-5.5% by weight.

47. System according to claim 45, wherein said pretreatment reactor and/or said at least one hydrolysis reactor are made of stainless steel.

48. Apparatus for impregnation of a cellulosic biomass under the usage of an acidic impregnation fluid of pH 0.5-5.5 comprising recycled and an optionally purified stillage, to obtain a impregnated cellulosic biomass in a process for production of ethanol liquid from said cellulosic biomass, said process further comprising acidic hydrolysis of said impregnated biomass to obtain a hydrolysate, fermentation of said hydrolysate to obtain a fermentation broth, distillation of said fermentation broth to provide said ethanol liquid and said stillage, and optionally, purification of said stillage, wherein the parts of said apparatus that contain said impregnation fluid consist of a material comprising iron in an amount of at least 50% by weight, chromium in an amount of 10.5-30% by weight, nickel in an amount of 2.5-29% by weight and molybdenum in an amount of 0-7% by weight.

49. Apparatus according to claim 48, further comprising a mixer adapted to mix said optionally purified stillage with an acid to produce said acidic impregnation fluid and an inlet in connection with said mixer adapted to receive said acidic impregnation fluid.

50. Method for the containment of an acidic impregnation fluid comprising a stillage from a distillation of a cellulosic fermentation broth derived from a cellulosic biomass, said impregnation fluid having a pH of 0.5-5.5, by means of which impregnation fluid a cellulosic biomass is impregnated at a temperature of 20-150° C., a pressure of 1-5 bar and a time of 1-120 minutes, wherein at least part of the container material being in contact with the acidic impregnation fluid comprises iron in an amount of at least 50% by weight, chromium in an amount of 10.5-30% by weight, nickel in an amount of 2.5-29% by weight and molybdenum in an amount of 0-7% by weight.
